(19) United States
(12) Patent Application Publication
(10) Pub. No.: US 2022/0081667 A1
(43) Pub. Date: Mar. 17, 2022

(54) BLOOD CIRCULATION FOR CULTURE GROWTH

(71) Applicant: Hewlett-Packard Development Company, L.P., Spring, TX (US)

(72) Inventors: Milo Overbay, Corvallis, OR (US); Viktor Shkolnikov, Palo Alto, CA (US)

(73) Assignee: Hewlett-Packard Development Company, L.P., Spring, TX (US)

(21) Appl. No.: 17/420,040

(22) PCT Filed: Jan. 18, 2019
(86) PCT No.: PCT/US2019/014191

§ 371 Date: Jun. 30, 2021

(51) Int. Cl. C12M 1/00; C12M 1/34; C12M 1/36

(52) U.S. Cl. CPC C12M 29/18; C12M 43/00; C12M 41/12; C12M 41/32; C12M 41/48; C12M 41/26; C12M 23/20

(57) ABSTRACT

Blood being circulated to mimic the environment it naturally inhabits increases growth of cultures including bacteria for analysis. The present disclosure presents examples of a device, method, computer-readable medium, and other techniques for simulating a natural environment for blood. The techniques may include a channel with a hydrophobic interior surface and a fluid mover to encourage continuous fluid flow through the channel. The techniques may further include a gas exchange opening of the channel that exposes a fluid to gas outside of the channel.

### BACKGROUND

A growth medium or culture medium is a solid, liquid or semi-solid designed to support the growth of microorganisms or cells, or small plants and mosses. Different types of media are used for growing different types of cells. Some media contain the nutrients required to support the growth of a wide variety of organisms.

### DESCRIPTION OF THE DRAWINGS

Certain examples are described in the following detailed description and in reference to the drawings, in which:

FIG. 1 is a block diagram of an example circulation system for blood circulation on a die;

FIG. 2 is a block diagram of an example channel layout on a die;

FIG. 3 is a block diagram of an example fluid flow path through a channel;

FIG. 4 is a block diagram of an example incubator channel stack;

FIG. 5 is a flowchart of an example fluid circulation ring;

FIG. 6 is a block diagram of an example computing system for culturing target microorganisms;

FIG. 7 is a flowchart of an example method for culturing target microorganisms; and

FIG. 8 is a block diagram of an example non-transitory, computer-readable medium including instructions to direct a processor for culturing target microorganisms.

### DETAILED DESCRIPTION

Many forms of bacteria are not able to be cultured under standard laboratory conditions. Culturing of bacteria enables further testing, identification, and production of bacterial or anti-bacterial products. A large number of bacteria and other living microorganisms thrive in the human body, including in the bloodstream where bacteria can causes bacteremia and sepsis.

The present disclosure relates to a fluid circulation device that can be applied in many ways but can be readily understood in the context of bacterial culture grown in blood samples. As an illustration, in order to provide proper treatment for bacteremia and sepsis, bacteria in blood needs to be identified, so that proper antibiotics can be given to a patient. Rather than provide broader stronger antibiotic that can lead to unintended side effects and promotion of antibiotic resistant strains of bacteria, well identified bacteria can be treated with a much narrower spectrum of antibiotic. In order to identify a bacteria, it must first be grown, or cultured to the point where identifying tests can be performed. Several issues can arise in the culturing and identification of the specific bacterial strain including that current bacterial culture mediums poorly simulate the blood environment as these mediums are often stagnant and has many unmet conditions of a regulated bloodstream.

It is also well known that whole blood rapidly degrades ex-vivo. One reason includes that blood viability for bacterial culturing degrades a result of settling of the cells and the stress that the cells experience associated with settling. Blood quality may also degrade due to changes in pH, propensity to coagulate, and depletion of energy sources such as glucose.

The present techniques show a microfluidic device which cultures the bacteria directly in sample blood in a more lifelike environment to both preserve the blood quality and preserve the target environment for the bacterial growth targeted for identification. Using these techniques aids in providing the correct environment both for the blood as a tissue to survive, and a proper environment for the difficult to culture bacteria to thrive and increase their numbers. The increased amount of bacteria culture aids in easier classification by standard bacterial classification and antibiotic susceptibility techniques.

These techniques may be implemented on a single component silicon die or microchip. The chip or die may include components and elements to maintain proper pH and utilize super hydrophobic, blood-repellent surfaces to retard blood coagulation. The chip or die may also provide glucose exchange and continuously circulates blood cells as to avoid settling and associated stress. As a note, coagulation and settling are two separate phenomenon. Settling referring to the physical separation and stagnation of the components in the blood particularly in a lack of movement of the blood. Coagulation referring the binding of blood components to one another.

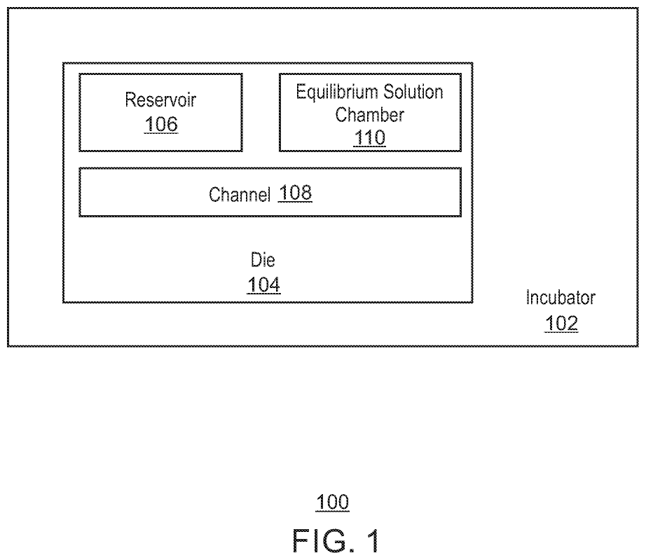

FIG. 1 is a block diagram of an example circulation system 100 for blood circulation on a die. The items shown may be connected to allow fluid to flow between them or sealed so that gas or fluid may not escape unless through a provided opening. The specific layout shown provides an example for a possible relationship of these elements being a part of a larger component or may indicate that they inside of the component.

The circulation system 100 includes an incubator 102. The incubator 102 may be a temperature preserving space. The incubator 102 may regulate temperature inside the incubator 102. The incubator 102 may also be a gas-tight chamber that maintains a designated internal atmosphere. In an example the internal atmosphere may be maintained at a constant oxygen content, nitrogen content, carbon dioxide content. The regulation of the atmospheric content may be by internal balances, internal controls, introduction of a particular gas into the incubator from within the incubator or introduction of a particular gas from outside the incubator 102. The humidity, or water content, of the internal atmosphere of the incubator 102 can also be regulated. In an example a humidifier or dehumidifier may be used within the walls of the incubator 102. A humidifier or dehumidifier may be used outside the walls of the incubator. Humidifiers and dehumidifiers include both devices and dehumidifying compounds. Gas with a known humidity may be introduced into the chamber to reach or maintain a preset humidity.

Within the incubator 102, a die 104 may be used to circulate a fluid such as blood. The die 104 can be a silicon die, a non-silicon die embedded with silicon components or communication connections. In an example, the die can be constructed with electrically conductive material. The die surface can be used to mount various components. A reservoir 106 may be disposed on the die 104. In an example, the reservoir 106 may be mounted or bonded onto the die 104. The reservoir 106 may be a smaller reservoir intended to hold an amount of fluid. The amount of fluid may correspond to the volume maximum of the fluid carrying areas of the die 104. The reservoir 106 may be a detached reservoir 106 that provides fluid, such as blood, to the die 104 for circulation. In an example, there can be a stir bar or other agitating component to avoid settling of the fluid in the reservoir 106. In an example, the reservoir 106 can move itself in order to avoid settling of components in the fluid. In an example the reservoir 106 is agitated through a haptic feedback component mounted on the die 104. The reservoir 106 may be located inside the incubator 102 or outside the incubator 102. The reservoir 106 may transport the fluid into the incubator 102 and/or to the die 104 if the reservoir 106 is located out of the incubator 102 or off the die 104, respectively.

Fluid provided by the reservoir 106 may travel through a channel 108. The channel can be cut out of the die 104 outlining a fluid flow path for the fluid to travel. The channel 108 can be guided by the shape of the die 104 and travel both in and out of a die 104 in a number of ways. In an example, the channel 108 may be on a single side of the die 104. The channel 108 may also pass through the die 104, be on both sides of the die 104, or any combination of these channel locations. The channel path may be coated in a material. The material may be an epoxy such as the epoxy resin known as Su8 or other compounds comprising epoxy groups. The material may be naturally hydrophobic or able to hold a hydrophobic coating. The material itself may form the entire channel and be hydrophobic, such as polytetrafluorethylene (PTFE). The material used may also be hydrophobic on the surface such as polypropylene formed inside another polymer. The material channel coating can be a lower part of a channel with a cover, hat, or top half of a channel missing, accordingly, a top hat later can be added to complete the enclosure of the channel in order to define a fluid flow path. The material of the channel can be hydrophobic. In an example, the interior surface is hydrophobic and the exterior of the channel is not hydrophobic. The interior surface of the channel 108 can be made hydrophobic by a coating applied to the interior surface of the channel 108. The interior surface of the channel 108 can be made hydrophobic by a coating applied to the interior surface of the channel before the channel is assembled or manufactured. The hydrophobic nature of the interior surface of the channel reduces blood coagulation and increases similarity to the arteries and veins of a living thing.

The interior surface of the channel 108 can be coated with ALD deposited and etched titania or alumina to form a nanoporous layer. This layer may repel blood and reduce coagulation. Some small amounts of anticoagulants, such as heparin, may be added to further reduce coagulation. Alternate surface treatments to keep the blood from adhering include treating the surface with a hydrophilic self-assembled monolayer (SAM) to retain water at the surface and reduce hydrophobic proteins. Another surface treatment method enabled by these techniques includes pre-coating the surface with a preferential plasma protein such as albumin which is inert to platelet adhesion and activation. Channels themselves may be fabricated out of plastics, such as cyclic polymers, such as COC, Su8 on glass, SU8 on plastic. The channels may be 3d print formed as pure plastics or a plastics with embedded electrodes for doing simple resistive heating. Resistive heating can include the use of a temperature sensitive resistor to provide feedback to control the amount of heating provided.

Alternatively, the microfluidic layers to form the channel 108 and loops may be embossed in an overmold material, while strategically placed Si dies provide pumping for nutrient injection into the fluid or sensing of temperature, pH, and other fluid conditions such as sugar content and others.

The channel 108 may be connected to an equilibrium solution chamber 110. The connection may be made using an arm or aperture of the channel 108. The equilibrium solution chamber 110 can be a glucose storage compartment. The glucose storage compartment can glucose in a solution that can be controlled in the concentration. As the concentration of glucose in the equilibrium solution chamber 110 changes, the connection of the equilibrium solution chamber 110 to the channel 108 enables the environment of fluid inside the channel 108 to be regulated. The environment of fluid inside the channel 108 may also be buffered or maintained depending on the specific solutions being used. In an example, the equilibrium solution chamber 110 contains at least one of glucose, salt, or other pH solutions. In an example, the equilibrium solution chamber 110 includes glucose in an isotonic solution at a set concentration. The equilibrium solution chamber 110 may be connected to the channel 108 through a semi-permeable membrane, a pump, an unimpeded but very small opening joining the channels, or other connection method allowing at least some form of particle exchange between the fluid in the channel and the fluid in the equilibrium solution chamber 110.

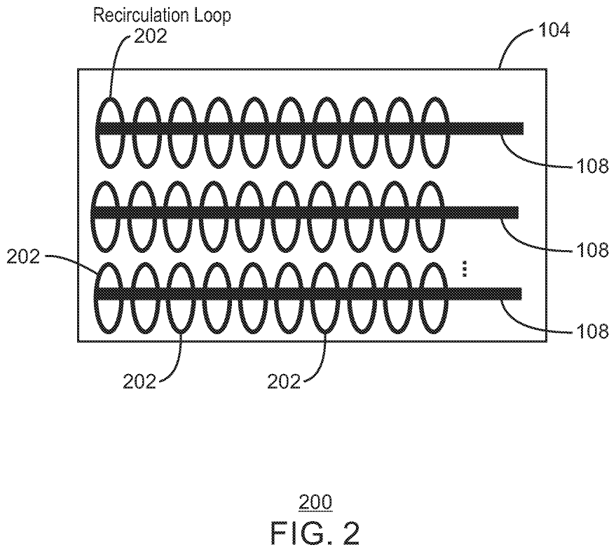

FIG. 2 is a block diagram of an example channel layout 200 on a die. Like numbered items are as described with respect to FIG. 1. The channel layout 200 shown provides one example layout.

The channel layout 200 of FIG. 2 shows a more detailed view of the die 104 of FIG. 1. Mounted on the die may be a channel 108 or a number of channels 108. In examples with a number of channels, each channel 108 may be laid out with a central channel vein running parallel to the other central channel veins. In an example, the die 104 of the channel layout 200 may be showing a single layer of the die 104 and another level of the die 104 may include an additional channel 108 group. In an example, each channel 108 is connected to one another. In an example, each channel may be separated from each other to isolate the fluid contained in each channel. In an example, a number of channels may be mounted on a single die and each channel may be fluidicly isolated from the other channels on the same die. Using multiple isolated channels on the same die enables multiple fluid compositions to be tested at the same time.

In an example, each channel 108 can be used for blood flow. The blood may be flowing to more closely simulate blood movement in a living organism. The continuous movement reduces coagulation of the blood. The more similar environment enables more accurate duplication of conditions for running tests on the blood. The more similar environment for the blood, can improve the yield of bacterial cultures found to grow best in blood. The bacterial cultures may not grow as well in coagulating or stagnant, unoxygenated blood at the wrong pH or glucose content. Accordingly, the ability to maintain circulation of the blood in a controlled environment increases the rate of growth and similarity of growth of these target microorganisms when compared to less lifelike culture mediums. In an example, the channel could include an inlet where stimulants could be injected into the fluid such as growth promoting hormones, additives for promoting metabolic pathways of the samples of bacteria, fungal, and viral cells, as relevant for the sample. To that effect, while bacterial cultures are focused on in these techniques, they may also be used to provide medium for growing and identifying fungal and viral growths.

To improve similarity to a living creature circulatory system, the channel 108 may include a loop 202 branching off form the main passageway of the channel 108. A channel 108 may include a single loop or a number of loops. The number of loops may all be to one side of a channel or on opposite sides of the channel 108. The loops may direct fluid flow in directions that are parallel to the surface of the channel mounting surface of the die 104. In an example, the loops are recirculation loops made of the same material as the channel 108. The loops can have a smaller internal diameter compared to the internal diameter of the channel 108. The loops as smaller passageways for fluid, such as blood, can better simulate capillaries, and varied speed of fluid flow similar to variations of fluid flow speed inside a body.

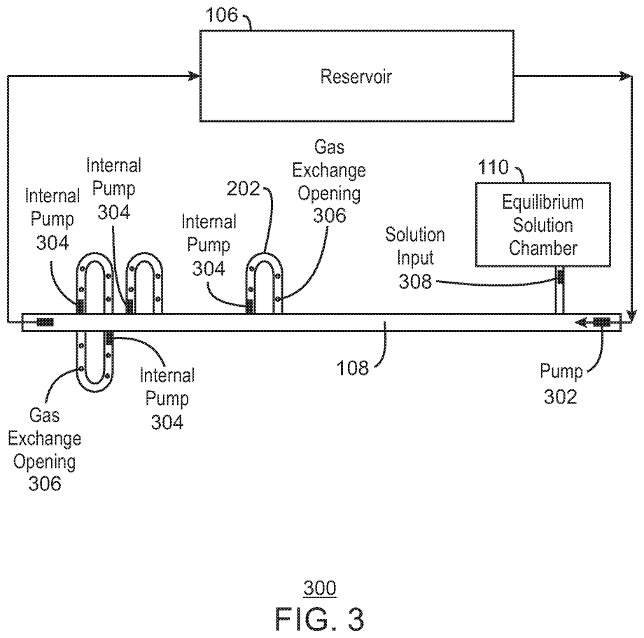

FIG. 3 is a block diagram of an example fluid flow path 300 through a channel. Like numbered items are as described with respect to FIG. 1 and FIG. 2.

The fluid flow path 300 shows a pump 302 present in the fluid flow path 300 drawing fluid from the reservoir 106 and into the channel 108. The pump 302 can be an inertial pump, an external pump, a powered pump, and other suitable pumps used in microfluidics. An external pump may be considered external depending on the pump location relative to an incubator 102, whether the pump is internal or external to the incubator 102. An external pump may be considered external depending on whether the pump driving force is generated inside the channel 108 or outside the channel 108 as well as on the die 104 or off the die 104.

The fluid flow path 300 can include an internal pump 304 that is inside the loop 202. In an example, the internal pump 304 is internal because it is within the channel 108 or within the recirculation loop 202. In an example, the pump 302 may pulse between a slow speed and a fast speed, or between no movement and movement in order to simulate a heartbeat rhythm.

A loop 202 may include an exit branch diverting fluid away from the channel 108 and a reentry branch where fluid rejoins the channel 108. In an example the internal pump 304 can be located in an exit branch or a reentry branch. In an example the internal pump 304 may be located in a downstream or upstream position relative to the fluid flow direction in the channel 108. In an example, a first internal pump 304 in a first recirculation loop 202 may be positioned in a downstream position and a second internal pump 304 in a second recirculation loop 202 may be positioned in an upstream position where the branches of the first and second recirculation loops are facing each other on opposite sides of the channel 108.

The loop 202 may include a gas-exchange opening 306. The gas exchange opening 306 may be an opening in the recirculation loop 202 that allow gas-exchange to occur between the gas in the incubator and the dissolved gases in the fluid being circulated. In an example, carbon-dioxide, oxygen, and water may be exchanged through the gas-exchange opening 306. In an example, the gas-exchange opening 306 are covered by membranes with specific permeability to allow specific particles to pass through, but not others. In an example, a membrane may allow only oxygen to pass through the membrane. The gas-exchange opening 306 may have a selective port or may be an open port. The gas-exchange opening 306 may be an open port with a size smaller than the surface tension of the fluid so that the fluid cannot escape through the opening. The gas-exchange opening 306 can be smaller than six micrometers to allow gas to pass through the opening while preventing particles in blood from escaping through the gas-exchange opening 306.

The equilibrium solution chamber 110 can include a solution for maintaining fluid equilibrium. In an example, the equilibrium solution chamber 110 may include an isotonic glucose solution. The solution in the equilibrium solution chamber 110 may be pumped directly into the channel 108 using a solution input 308. The solution input 308 may also be covered by a membrane with pass-through sizes corresponding to desired particles. In an example, the equilibrium solution chamber 110 may be used to maintain a preset pH for the fluid inside the channel 108.

In an example, the channel can also including sensing or monitoring components to identify concentrations of particles or growth of bacteria and other cultures. In an example, the sensing or monitoring may be through observing production of metabolites such as bacterial toxins. The observation may be made by via chem-field effect transistors (FETs) embedded into the silicon die. Sensing and observing may also take place via the tracking and comparison of scattering profiles of particles flowing in the gas exchange channels over time. For example, a scattering profile include any optical analysis method for identifying the components and properties of the analyzed tissue. In this context, if scattering profiles were identified for the gas exchanged outside of the gas-exchange openings in the recirculation loops, then this change could correspond to the new presence of new small particles such as bacteria. Accordingly, the present techniques include sensors and observation components both in the channel and outside the gas-exchange openings.

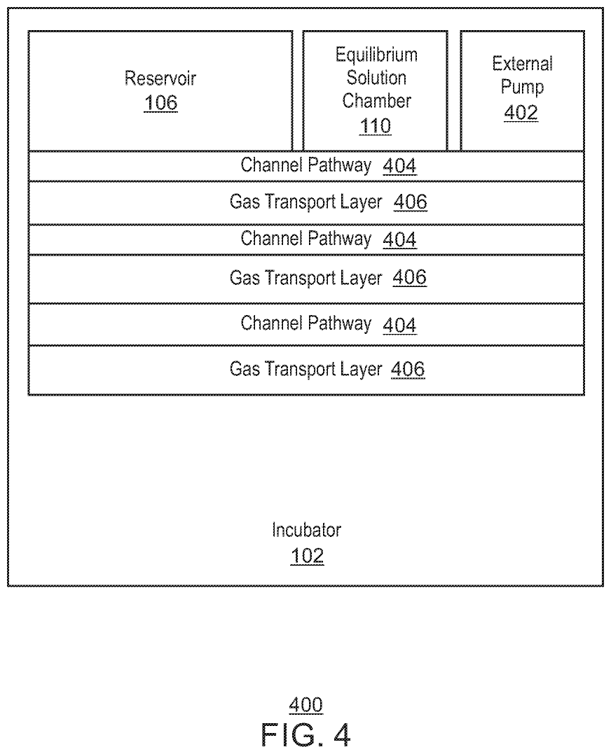

FIG. 4 is a block diagram of an example incubator channel stack 400. Like numbered items are as described with regard to FIG. 1. The channel stack 400 shows in part how channels can be arrange, either on the die or off the die to enable fluid flow. The incubator channel stack 400 also provides additional conditions more similar to circulation of blood in a body. In an example, the incubator channel stack 400 can include an external pump 402. As discussed above, the external pump 402 may be external as it may not be mounted on the die 104, outside the incubator 102, or power generation takes place outside of the channel 108 and translated to a pumping mechanism or material inside the channel 108. In some examples, the external pump 402 may not be present. The external pump 402 may also be a syringe pump, pressure regulator, centrifugal pumps, or other fluidic pump. The external pump can apply pressure on the reservoir holding an equilibrium solution, such as glucose solution.

For example, if the channel 108 for blood circulation was hosted entirely on a chip, then an external pump to move the fluid may not be necessary. If the channel traveled from a reservoir or inlet outside of the die 104 or outside of the incubator 102 into the die 104 or incubator 102 and then back, then the external pump 402 is included to move the fluid.

The channel stack 400 shows channel pathways 404 which include both the channel 108 and the loop 202 or plurality of recirculation loops. As shown there may be one or more channel pathways 404 in order to increase throughput and total volume of the system as well as surface area of the channel in the system. Between multiple channels can be gas transport layers 406. Gas transport layers may be spaces between the channels located inside the incubator, the spaces to allow gas or atmosphere in the incubator to interact with the fluid in the channel pathway 404. In an example, the gas transport layer 406 includes a fan in order to move air through the gas transport layer 406. The moving air may better simulate breathing and the gas-exchange enabled by moving gas increasing the gas particle availability to fluids at the gas-exchange openings.

In an example the channel stack 400 may be arranged with similar alternating spacing between channel pathways 404 and gas transport layers 406 with a difference being that all channel pathways 404 would exist on a die 104. The channel pathways could be made of material of the die or mountable on the die with hydrophobic properties to reduce coagulation of blood on those surfaces.

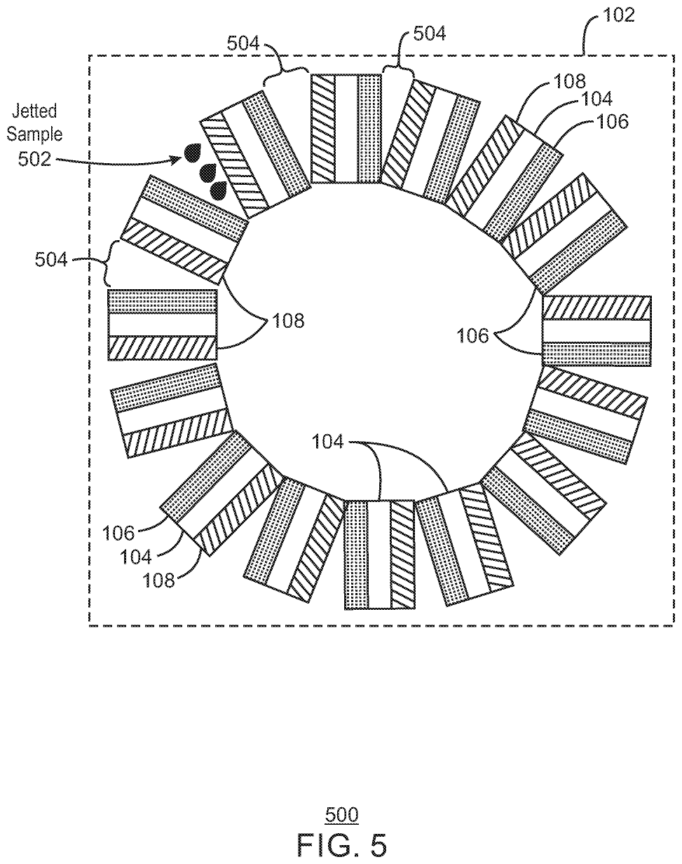

FIG. 5 is a flowchart of an example fluid circulation ring 500. Like numbered items are as described with respect to FIG. 1. The fluid circulation ring shows a layout for fluid circulation involving a number of reservoirs, dies, and channels. The fluid is jetted or propelled from one channel to another reservoir through gas, thus exposing the fluid to the maintained atmosphere inside the incubator 102. The exposure of the fluid to the atmosphere allows gas-exchange to occur and keeps the fluid moving. The gas-exchange and continuous motion can prevent coagulation and encourage the growth of target microorganism such as bacterial cultures that grow in living blood. Any growth that is naturally found in living blood may have a better chance of survival in environments where the blood is kept moving and can experience gas-exchange.

The shape of any component shown may be adjusted to suit the fluid flow direction of the fluid circulation ring 500 desired. In an example, there may be only two reservoirs and two channels each jetting or propelling fluid from a first to a second and back again. As long as there is at least one gap where the fluid particles pass through the gas atmosphere of the incubator, the channel of each component does not need to include recirculation loops or loop based gas-exchange openings.

In an example in the fluid circulation ring 500, a sample may be jetted from an ink jet component in a channel 108 towards a subsequent reservoir 106. The jetting may be done by microfluidics propulsion, a thermal inkjet resistor, or piezo element. Using these elements, creates the momentum on the jetted sample 502 such as droplets of fluid, such as blood. The jetting through the gas of the incubator to the reservoir enables gas exchange enabling cells and bacterial in the fluid to maintain their viability. In a thermal inkjet resistor, the temperature of the resistor acts on a small layer of the blood, quickly boiling it and from the quick expansion from the liquid state to the gas state, pushes all surrounding liquid away from the resistor. When the fluid mover is a thermal inkjet resistor or piezo element, the gas exchange opening can be the space 504 between the thermal inkjet resistor and a reservoir in a path of the channel. In an example, the space 504 may be present between each channel and reservoir 106 in the fluid circulation ring 500. The fluid circulation ring may restrict itself to one space 504 between a channel and reservoir enabling gas-exchange. Limiting the number of spaces 504 between elements can decrease size and chance for leaks.

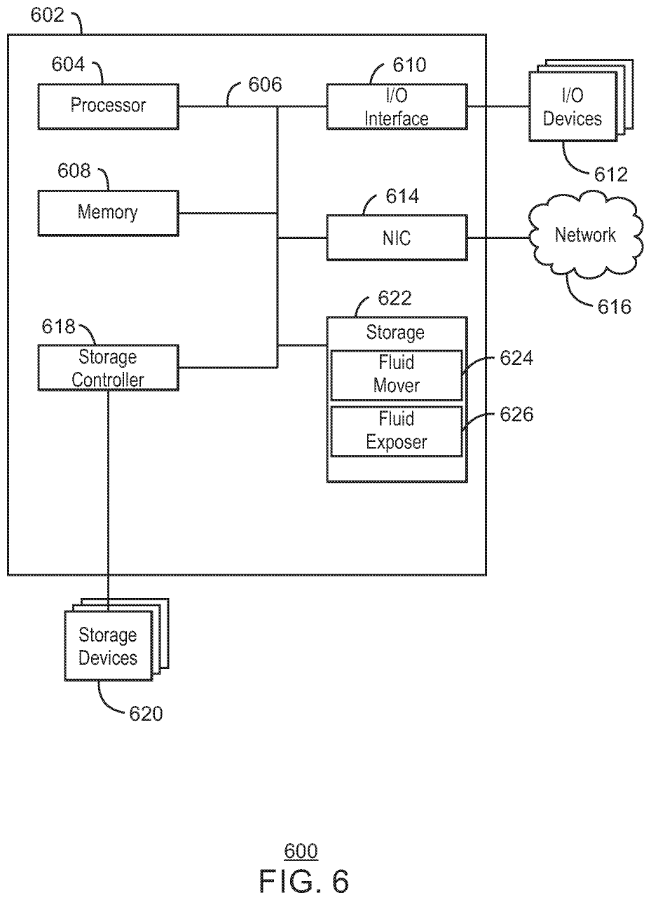

FIG. 6 is a block diagram of an example computing system 600 for blood circulation as an environment for culturing target microorganisms. The system for blood circulation as an environment for culturing target microorganisms can be a standalone computing device 602 for blood circulation and growth analysis. This computing device 602 can include a desktop computer, laptop, tablet, mobile phone, smart device, printer hub, printer controller, or other computing devices. The system 600 for blood circulation as an environment for culturing target microorganisms includes at least one processor 604. The processor 604 can be a single core processor, a multicore processor, a processor cluster, and the like. The processor 604 can may include a graphics processing unit (GPU), an application specific integrated circuit (ASIC), or a field programmable gate array (FPGA), or any combination thereof to implement video processing. The processor 604 can be coupled to other units through a bus 606. The bus 606 can include peripheral component interconnect (PCI) or peripheral component interconnect express (PC1e) interconnects, Peripheral Component Interconnect eXtended (PCIx), or any number of other suitable technologies for transmitting information. In an example, the computing device 602 may be within an incubator chamber or conductively connected to components within an incubator chamber.

The computing device 602 can be linked through the bus 606 to a memory 608. The system memory 608 can include random access memory (RAM), including volatile memory such as static random-access memory (SRAM) and dynamic random-access memory (DRAM). The system memory 608 can include directly addressable non-volatile memory, such as resistive random-access memory (RRAM), phase-change memory (PCRAM), Memristor, Magnetoresistive random-access memory, (MRAM), Spin-transfer torque Random Access Memory (STTRAM), and any other suitable memory that can be used to provide computers with persistent memory.

The processor 604 may be coupled through the bus 606 to an input/output (I/O) interface 610. The I/O interface 610 may be coupled to any suitable type of I/O devices 612, including input devices, such as a mouse, touch screen, keyboard, display, VR/AR controllers through body movement detection cameras, handheld controllers and the like. The I/O devices 612 may be output devices such as a display, VR/AR goggles, a projector, and the like.

The computing device 602 can include a network interface controller (NIC) 614, for connecting the computing device 602 to a network 616. In some examples, the network 616 can be an enterprise server network, a storage area network (SAN), a local area network (LAN), a wide-area network (WAN), or the Internet, for example. The processor 604 can be coupled to a storage controller 618, which may be coupled to one or more storage devices 620, such as a storage disk, a solid state drive, an array of storage disks, or a network attached storage appliance, among others.

The computing device 602 can include a non-transitory, computer-readable storage media, such as a storage 622 for the long-term storage of data, including the operating system programs and user file data. The storage 622 can include local storage in a hard disk or other non-volatile storage elements. While generally system information may be stored on the storage 622, in this computing device 602, the program data can be stored in the memory 608. The storage 622 may store instructions that may be executed by the processor 604 to perform a task.

The storage 622 can include a fluid mover 624 to instruct the computer device to move a fluid continuously through a channel with a hydrophobic interior. The fluid movement may be carried out via the I/O devices which can include ink jet resistors. The fluid movement may be instructed to components manipulating fluid like blood in a channel. In an example, the channel includes a hydrophobic interior surface.

The storage 622 can include a fluid exposer 626 to direct the fluid in a path to be exposed to gas outside of the channel through a gas exchange opening of the channel. In an example, the channel includes a metabolite detector embedded into a channel surface. The metabolite detector may be used to sense or monitor the growth of bacteria, for example by observing production of certain metabolites like bacterial toxins. The observation of bacterial toxins may be made by chemical-field effect transistors embedded into the computing device or a silicon die of the channel.

The gas exchange opening may be located on a loop shaped to steer fluid flow away from the channel and then back into the channel. The size of the gas exchange opening may be smaller than six micrometers. The size of the gas exchange may be smaller than one hundred micrometers. The size of the gas exchange may be in the range of one to five micrometers. The size of the gas exchange may be smaller than one micrometer. The size of the gas exchange may be between twenty and ten micrometers. In an example, the fluid mover is a thermal inkjet resistor or piezo element. When the fluid mover is a thermal inkjet resistor or piezo element, the gas exchange opening can be the space between the thermal inkjet resistor and a reservoir in a path of the channel.

The system 600 may include a gas exchange opening that exposes a fluid inside the channel to moving gas. In an example the moving gas is due to a gas moving element to direct gas movement over an element. In an example, the gas exchange opening is covered by a gas-permeable membrane. The system 600 may also include a glucose storage compartment attached to the channel allowing exchange of glucose with the channel. In an example, the system 600 includes a particle detector in the gas exchange opening to report the gas scatter profile at a number of times. For example, a particle detector could observe the scattering profile of particles flowing in the gas exchange over time. Using a particle detector over time could suggest bacterial growth based on the change in the overall profile of the scattering profile due to the new presence of new small particles over time. The system can include a toxin removing balancer to selectively remove identified toxins from the fluid and balance the fluid to contain identified metrics. The toxin removing balancer can make use fluid exchange techniques to remove bacteria toxins. For example, a toxin removing balancer could use dialysate to remove human toxins. The toxin removing balancer could also rebalance electrolytes and minerals desired to be in the fluid by addition or removal.

It is to be understood that the block diagram of FIG. 6 is not intended to indicate that the computing device 602 is to include all of the components shown in FIG. 6. Rather, the computing device 602 can include fewer or additional components not illustrated in FIG. 6.

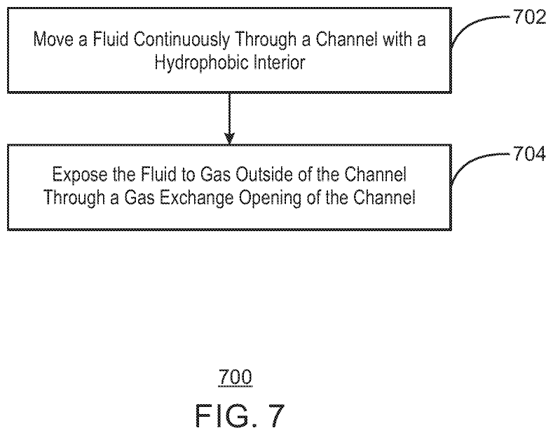

FIG. 7 is a flowchart of an example method 700 for blood circulation as an environment for culturing target microorganisms. At block 702, the method 700 moves a fluid continuously through a channel with a hydrophobic interior. The fluid movement may be carried out via the I/O devices which can include ink jet resistors. The fluid movement may be instructed to components manipulating fluid like blood in a channel. In an example, the channel includes a hydrophobic interior surface.

For the method 700, the channel can include a metabolite detector embedded into a channel surface. The metabolite detector may be used to sense or monitor the growth of bacteria, for example by observing production of certain metabolites like bacterial toxins. The observation of bacterial toxins may be made by chemical-field effect transistors embedded into the computing device or a silicon die of the channel.

At block 704, the method exposes the fluid to gas outside of the channel through a gas exchange opening of the channel. The gas exchange opening may be located on a loop shaped to steer fluid flow away from the channel and then back into the channel. The size of the gas exchange opening may be smaller than six micrometers. The size of the gas exchange may be smaller than one hundred micrometers. The size of the gas exchange may be in the range of one to five micrometers. The size of the gas exchange may be smaller than one micrometer. The size of the gas exchange may be between twenty and ten micrometers. In an example, the fluid mover is a thermal inkjet resistor or piezo element. When the fluid mover is a thermal inkjet resistor or piezo element, the gas exchange opening can be the space between the thermal inkjet resistor and a reservoir in a path of the channel.

The method 700 may include exposing the fluid inside the channel to moving gas. In an example the moving gas is due to a gas moving element to direct gas movement over an element. In an example, the gas exchange opening is covered by a gas-permeable membrane. The method 700 may also include a step for exchanging glucose with a glucose storage compartment attached to the channel allowing exchange of glucose with the channel. In an example, the method 700 includes detection using a particle detector in the gas exchange opening to report the gas scatter profile at a number of times. For example, a particle detector could observe the scattering profile of particles flowing in the gas exchange over time. Using a particle detector over time could suggest bacterial growth based on the change in the overall profile of the scattering profile due to the new presence of new small particles over time.

It is to be understood that the block diagram of FIG. 7 is not intended to indicate that the method 700 is to include all of the actions shown in FIG. 7. Rather, the method 700 can include fewer or additional components not illustrated in FIG. 7.

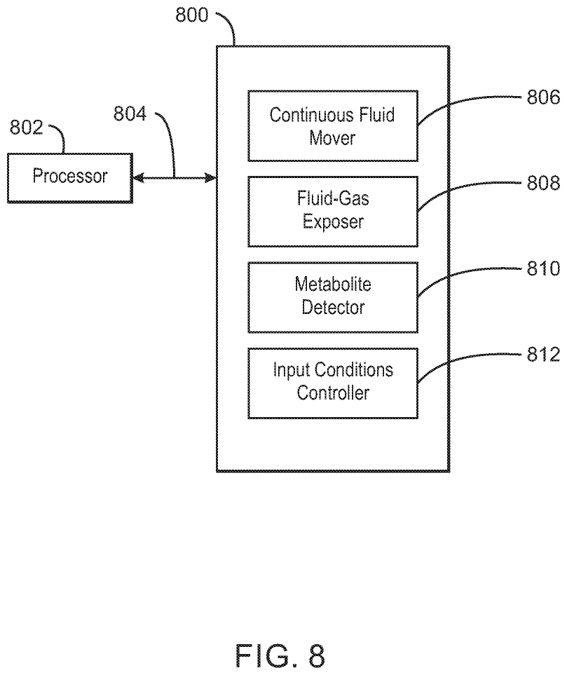

FIG. 8 is a block diagram of an example non-transitory, computer-readable medium 800 including instructions to direct a processor for blood circulation as an environment for culturing target microorganisms. The computer readable medium 800 may include the storage 622 or the memory 608 of FIG. 6 and other suitable formats readable by the computing device. The computer readable medium 800 can include the processor 802 to execute instructions received from the computer-readable medium 800. Instructions can be stored in the computer-readable medium 800. These instructions can direct the processor 802 for blood circulation as an environment for culturing target microorganisms. Instructions can be communicated over a bus 804 as electrical signals, light signals, or any other suitable means of communication for transmission of data in a similar computing environment.

The computer-readable medium 800 includes a continuous fluid mover 806 to instruct the computer device to move a fluid continuously through a channel with a hydrophobic interior. The fluid movement may be carried out via ink jet resistors or piezo elements. The fluid movement may be instructed to components manipulating fluid like blood in a channel. In an example, the channel includes a hydrophobic interior surface.

The computer-readable medium 800 includes a fluid-gas exposer 808 to direct the fluid in a path to be exposed to gas outside of the channel through a gas exchange opening of the channel. In an example, the channel includes a metabolite detector embedded into a channel surface. The metabolite detector may be used to sense or monitor the growth of bacteria, for example by observing production of certain metabolites like bacterial toxins. The observation of bacterial toxins may be made by chemical-field effect transistors embedded into the computing device or a silicon die of the channel.

The computer-readable medium 800 includes a metabolite detector 810 to detect a metabolite in the fluid with a detector disposed in the channel. The computer-readable medium 800 can include an input conditions controller 812 to adjust input conditions based on a presence of metabolite to achieve a target metabolite measurement. In an example, the input conditions are at least one of heat of the channel, glucose concentration of the fluid in the channel, salt concentration of the fluid in the channel, or pH of the fluid in the channel.

The gas exchange opening may be located on a loop shaped to steer fluid flow away from the channel and then back into the channel. The size of the gas exchange opening may be smaller than six micrometers. The size of the gas exchange may be smaller than one hundred micrometers. The size of the gas exchange may be in the range of one to five micrometers. The size of the gas exchange may be smaller than one micrometer. The size of the gas exchange may be between twenty and ten micrometers. In an example, the fluid mover is a thermal inkjet resistor or piezo element. When the fluid mover is a thermal inkjet resistor or piezo element, the gas exchange opening can be the space between the thermal inkjet resistor and a reservoir in a path of the channel.

The computer-readable medium 800 may instruct a processor to direct components of a device that include a gas exchange opening that exposes the fluid inside the channel to moving gas. In an example the moving gas is due to a gas moving element to direct gas movement over an element. In an example, the gas exchange opening is covered by a gas-permeable membrane. The computer-readable medium 800 may instruct a processor to direct components of a device that include a glucose storage compartment attached to the channel allowing exchange of glucose with the channel. In an example, the computer-readable medium 800 may instruct a processor to direct components of a device that includes a particle detector in the gas exchange opening to report the gas scatter profile at a number of times. For example, a particle detector could observe the scattering profile of particles flowing in the gas exchange over time. Using a particle detector over time could suggest bacterial growth based on the change in the overall profile of the scattering profile due to the new presence of new small particles over time.

It is to be understood that the block diagram of FIG. 8 is not intended to indicate that the computer-readable medium 800 is to include all of the components shown in FIG. 8. Rather, the computer-readable medium 800 can include fewer or additional components not illustrated in FIG. 8.

While the present techniques may be susceptible to various modifications and alternative forms, the techniques discussed above have been shown by way of example. It is to be understood that the technique is not intended to be limited to the particular examples disclosed herein. Indeed, the present techniques include all alternatives, modifications, and equivalents falling within the scope of the following claims.

What is claimed is:

1. A device for blood circulation as an environment for culturing target microorganisms comprising:
a channel with a hydrophobic interior surface;
a fluid mover to encourage continuous fluid flow through the channel; and
a gas exchange opening of the channel that exposes a fluid to gas outside of the channel.

2. The device of claim 1, wherein the gas exchange opening is located on a loop shaped to steer fluid flow away from the channel and then back into the channel.

3. The device of claim 1, wherein the gas exchange opening is smaller than six micrometers.

4. The device of claim1, wherein the fluid mover is a thermal inkjet resistor.

5. The device of claim 4, wherein the gas exchange opening is space between the thermal inkjet resistor and a reservoir in a path of the channel.

6. The device of claim 1, comprising a glucose storage compartment attached to the channel allowing exchange of glucose with the channel.

7. The device of claim 1, wherein the channel comprises a metabolite detector embedded into a die surface.

8. The device of claim 1, comprising a particle detector in the gas exchange opening to report a gas scatter profile at a plurality of times.

9. The device of claim 1, comprising a toxin removing balancer to selectively remove identified toxins from the fluid and balance the fluid to contain identified metrics.

10. The device of claim 1, wherein the gas exchange opening is covered by a gas-permeable membrane.

11. A method for blood circulation as an environment for culturing target microorganisms comprising:
moving a fluid continuously through a channel with a hydrophobic interior; and
expose the fluid to gas outside of the channel through a gas exchange opening of the channel.

12. The method of claim 11, wherein the gas exchange opening is located on a loop shaped to steer fluid flow away from the channel and then back into the channel.

13. The method of claim 11, wherein the gas exchange opening is smaller than six micrometers.

14. A computer readable medium for blood circulation as an environment for culturing target microorganisms comprising instructions that when executed on a processor instruct the processor to:
move a fluid continuously through a channel with a hydrophobic interior;
expose the fluid to gas outside of the channel through a gas exchange opening of the channel; and
detect a metabolite in the fluid with a detector disposed in the channel;
control input conditions based on a presence of metabolite to achieve a target metabolite measurement.

15. The computer readable medium of claim 14, wherein the input conditions are at least one of heat, glucose concentration, salt concentration, or pH.
